(19) United States
(12) Patent Application Publication
(10) Pub. No.: US 2022/0265485 A1
(43) Pub. Date: Aug. 25, 2022

(54) ABSORPTION SHORTS

(71) Applicant: Be-A Japan Inc., Tokyo (JP)

(72) Inventor: Kumi TAKAHASHI, Tokyo (JP)

(21) Appl. No.: 17/618,840

(22) PCT Filed: Jul. 28, 2020
(86) PCT No.: PCT/JP2020/028811

§ 371 Date: Dec. 13, 2021

(30) Foreign Application Priority Data
Apr. 24, 2020 (JP) 2020-077847

(51) Int. Cl. A61F 13/496; A61F 13/49

(52) U.S. Cl. CPC A61F 13/496; A61F 13/49017; A61F 13/505

(57) ABSTRACT

The present disclosure is an absorption shorts including a shorts body having a waist opening and a pair of leg openings, and an absorbent member. The absorption member has a pair of wing parts on the left and right sides in the portion corresponding to the crotch part. The absorption member is sewn to the shorts body at the front end part, the rear end part, and the wing part. According to the absorption shorts of the present disclosure, menstrual blood and urine can be absorbed. Moreover, it can be used repeatedly by washing.

### TECHNICAL FIELD

The present disclosure relates to absorption shorts.

### BACKGROUND ART

Patent Literature 1 discloses sanitary shorts that has an absorption performance capable of absorbing a liquid such as menstrual blood discharged from the body and can be washed and used repeatedly.

### PRIOR ART

### Patent Literature

Patent Literature 1: Japanese Patent No. 4073391

### SUMMARY OF THE INVENTION

### Technical Problem

The shorts described in Patent Literature 1 are shorts in which an absorption layer 7 and a leak-proof layer 8 are arranged on a crotch part 4 (elastic sheet 41) constituting a shorts body, so as to absorb menstrual blood and urine. The elastic sheet 41 constituting the crotch part 4, the absorption layer 7 and the leak-proof layer 8 have the same shape, and are integrated by suturing over the entire periphery. With such a configuration, when the shorts is washed by hand, it is difficult to wash the absorption layer 7 infiltrated with menstrual blood or urine.

Therefore, it is an object of the present disclosure to provide shorts that can absorb menstrual blood and urine and are easy to be washed by hand.

### Technical Solution

According to the present disclosure, there is provided an absorption shorts comprising a shorts body having a waist opening and a pair of leg openings, and an absorption member, wherein the absorption member has a pair of wing parts on the left and right sides at a portion corresponding to a crotch part, and the absorption member is sewn onto the shorts body at the wing part, and front and rear end parts of the absorption member.

### Advantageous Effects

According to the present disclosure, it is easy to wash the absorption part infiltrated with menstrual blood and urine by hand.

### BRIEF DESCRIPTION OF THE DRAWING

FIG. 1 is a development view of an absorption shorts according to an embodiment of the present disclosure.

FIG. 2 is a stereographic view of an absorption shorts according to an embodiment of the present disclosure.

FIGS. 3A, 3B, 3C, and 3D are exploded views of an absorption part according to an embodiment of the present disclosure.

FIG. 4 is a view showing a sewing part of an absorption part according to an embodiment of the present disclosure.

FIGS. 5A and 5B are cross-sectional views of an absorption part according to an embodiment of the present disclosure.

FIG. 6 is a development view of other absorption shorts according to an embodiment of the present disclosure.

FIGS. 7A and 7B are appearances of an absorption shorts according to an embodiment of the present disclosure.

### DETAILED DESCRIPTION OF THE EMBODIMENTS

The contents of the embodiment of the present disclosure will be listed and described. The present disclosure has the following configuration.

[Item 1]

An absorption shorts comprising:

a shorts body with a waist opening and a pair of leg openings, and

an absorption member,

wherein the absorption member is sewn onto the shorts body in a front end part, a rear end part, and left and right end parts of a portion corresponding to a crotch part.

[Item 2]

The absorption shorts as set forth in Item 1, wherein:

the absorption member is sewn onto the shorts body only at the front end part, the rear end part, and the left and right end parts of the portion corresponding to the crotch part.

[Item 3]

The absorption shorts as set forth in Items 1 or 2, wherein:

the absorption member has a pair of wing parts on the left and right sides in the portion corresponding to the crotch part, and is sewn onto the shorts body at the wing part.

[Item 4]

The absorption shorts as set forth in any one of Items 1 to 3, wherein:

the absorption member comprises

a first cloth member placed on a skin side, and

a second cloth member which is laminated below the first cloth member and has an absorption layer,

wherein the first cloth member and the second cloth member are not sewn onto at least a part of the left and right end parts.

[Item 5]

The absorption shorts as set forth in any one of Items 1 to 4, wherein:

the front end part and the rear end part of the absorption member are sewn onto the shorts body along a waist rubber of the shorts body, respectively.

[Item 6]

The absorption shorts as set forth in any one of Items 1 to 6, wherein:

the absorption member has a heat retaining part from the front end part to a lower abdomen.

[Item 7]

The absorption shorts as set forth in any one of Items 1 to 6, wherein:

the shorts body is comprising a substantially water-repellent fabric except the waist rubber as a whole.

### DETAILS OF EMBODIMENTS

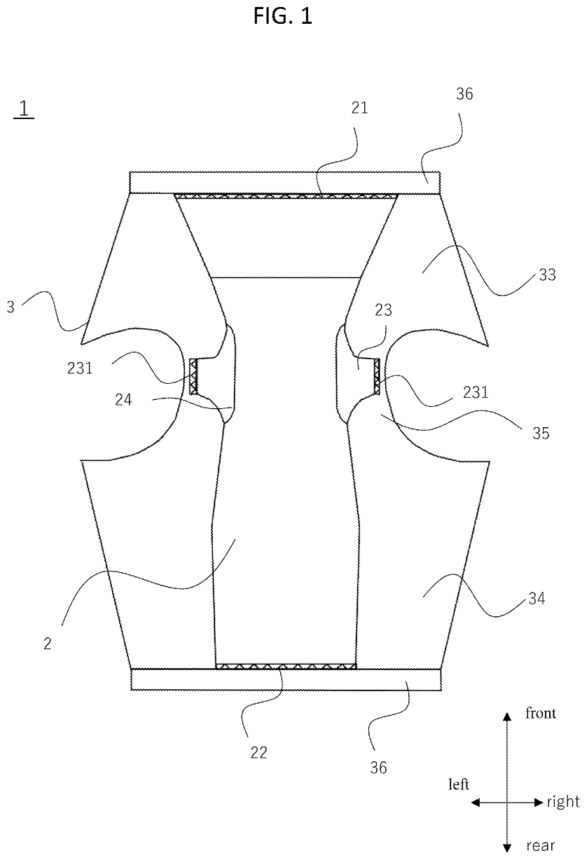

Hereinafter, embodiments of the present disclosure will be described with reference to the drawings. Also, as used herein, the terms “front”, “rear”, “left”, and “right” are used conforming 1s to the front/rear, left/right when wearing shorts, as shown in FIGS. 1 to 3.

FIG. 1 is a view in which the absorption shorts of the present disclosure are cut and developed at the left and right side parts. The absorption shorts are composed of a shorts body 3 and an absorption member 2 attached to the inside of the shorts body 3. Since the absorption shorts of the present disclosure include an absorption member 2 capable of absorbing and retaining menstrual blood and urine, clothes will not get soiled even without using sanitary napkins, incontinence pads, and the like. Further, the absorption shorts can be used repeatedly by laundering. Therefore, it is economical and safe even for those who are prone to skin irritation with commercially available sanitary napkins or the like. Further, if a wearer does not want to soil clothes, or on a day when the quantity of menstrual blood is large, the wearer can attach a sanitary napkin or an incontinence pad on the absorption member 2 or in the layer thereof for use.

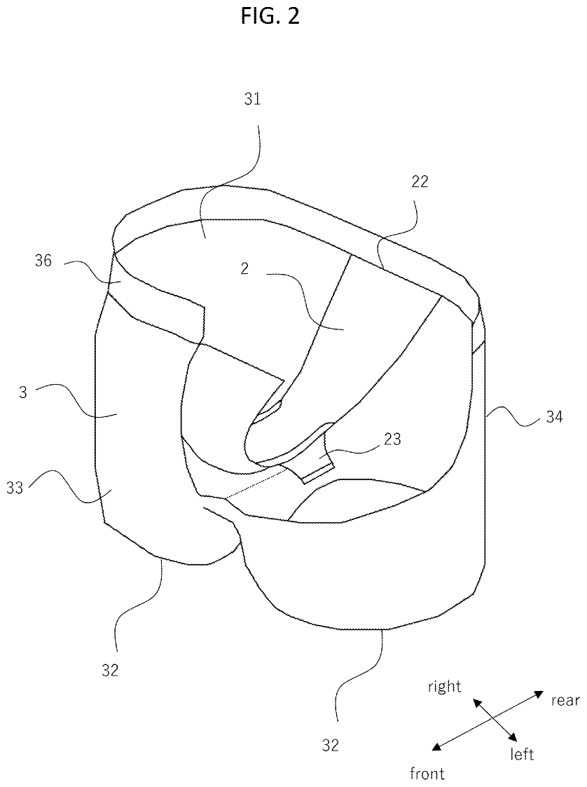

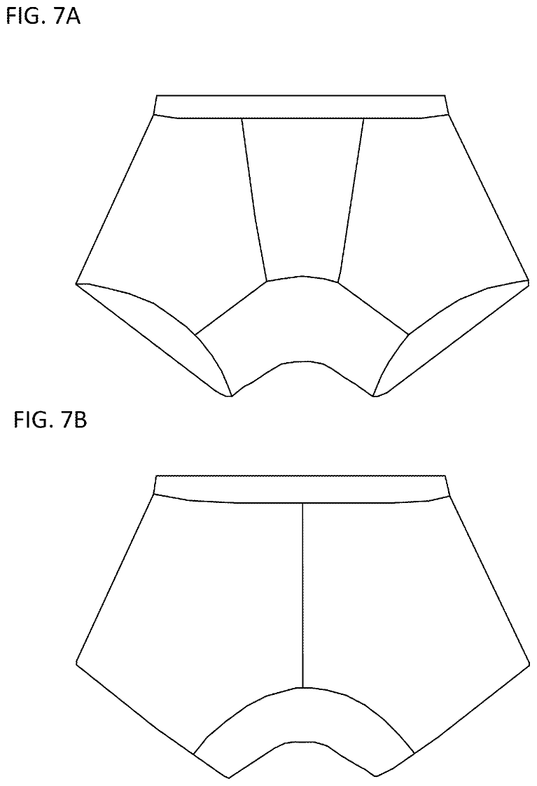

The shorts body 3 constitutes the shape of general shorts. The shorts of the present disclosure may be either for women or men, and the shape is not limited to the so-called boxer type as shown in FIGS. 2 and 7, and may have any shape. As shown in FIG. 2, the shorts body 3 includes a waist opening 31 and a pair of leg openings 32. A waist rubber 36 is coupled to the waist opening 31 along the end.

The shorts body 3 has a front body part 33, a back body part 34, and a crotch part 35 placed therebetween. The front body part, the back body part, and the crotch part mentioned herein do not necessarily refer to fabric parts, but to the area when the shorts body 3 is divided into three parts to the front and back. Therefore, the shape of the fabric parts when the shorts body 3 is to created is not particularly limited, and the fabric parts corresponding to the front center part, the crotch part, and the left and right parts may be sewn together as shown in FIG. 7. Fabric parts corresponding to the front body part, the back body part, and the crotch part may be sewn together.

The shorts body 3 is made of water-repellent fabric. The water-repellent fabric may be polyester, cotton, nylon, laminated fabric, or the like, and may be subjected to water repellent treatment. In particular, a blended fabric in which polyester and cotton are mixed at a predetermined ratio is preferable. As the shorts body 3 is made of a substantially water-repellent fabric except the waist rubber part as a whole, it does not stain clothes even when the liquid leaks from the absorption member 2.

The absorption member 2 is attached to the inside (skin side) of the shorts body 3. The absorption member 2 has a water-absorbing material, can absorb liquids discharged from the body such as menstrual blood and urine, and can prevent the shorts body 3 from seeping.

The absorption member 2 according to the embodiment of the present disclosure has an elongated shape in the front/back direction as a whole as shown in FIG. 1. A water-absorbing material of the absorption member 2 may be arranged on its entire surface, or may be arranged only on a part of it. The absorption member 2 has a front end part 21 and a rear end part 22, and is sewn and joined along the end part of the shorts body 3, specifically, the lower end part of the waist rubber 36. Further, the absorption member 2 may be sewn onto the shorts body 3 in the vicinity of the central part of the front/back direction and in the portion corresponding to the crotch part. When sewn onto the shorts body 3 in the vicinity of the central part, it is sewn onto the fabric of the shorts body 3 along the left and right end parts of the absorption member 2 over a predetermined length. Further, the absorption member 2 preferably includes a wing part 23 extending in the left/right direction. The wing part 23 has a width in the front/back direction, and the right wing part 23 has a wing end part 231 at the right end, and the left wing part 23 has a wing end part 231 at the left end. The wing part 23 is sewn to the crotch part of the shorts body 3. For example, it may be sewn onto the shorts body 3 in the wing end parts 231 at both wing parts 23.

The absorption member 2 of the present disclosure is joined to the fabric of the shorts body 3 only at the front end part 21 and the rear end part 22. Alternatively, in the vicinity of the central part in addition to the front end part 21 and the rear end part 22, particularly in the left and right 1s wing parts 23 when having wing parts 23, it is joined to the fabric of the shorts body 3 in the left and right wing parts 23. On the other hand, it is preferable that the other portion does not have a connecting part with the shorts body 3. Since most of the absorption member 2 is separated from the shorts body 3, when shorts are washed after absorbing liquid, the absorption member 2 can be pulled away from the shorts body 3, and the absorption member 2 can be washed by rubbing. On the other hand, since the front end part 21, the rear end part 22, and the wing part 23 are fixed to the shorts body 3, the absorption member 2 does not shift when worn. By providing the wing part 23, the movement of the crotch part during wearing is restricted, and it can be worn with greater peace of mind. The absorption member 2 is separated by the length of the wing part 23 during washing, thus facilitating washing.

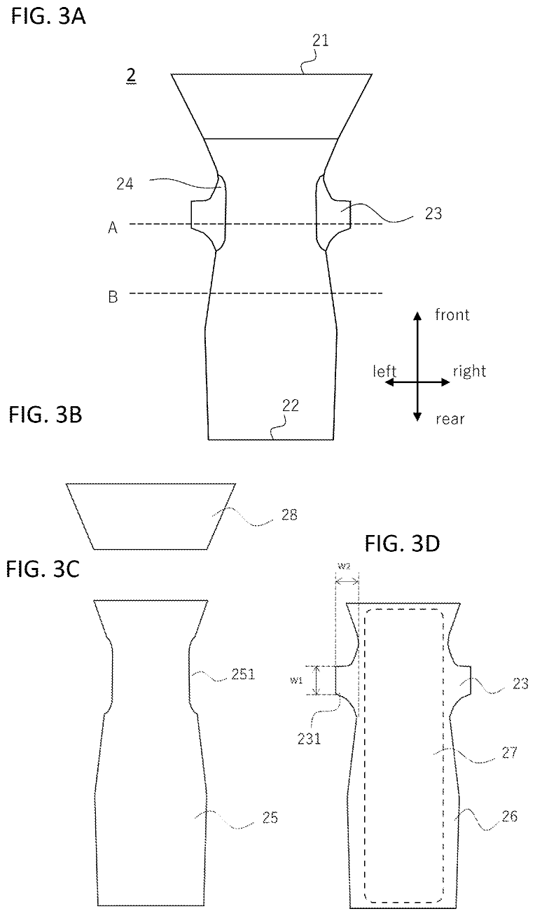

FIGS. 3A, 3B, 3C, and 3D show a specific structure of the absorption member 2. FIG. 3A is a view showing the entire absorption member 2. The absorption member 2 has a substantially band-like shape elongated from the front end part 21 to the rear end part 22. In particular, the predetermined length part from the front end part 21 to the center corresponding to the lower crotch part may be gradually narrowed in width (trapezoid with the front end part 21 as the long side). The width of the front end part 21 of the absorption member 2 depends on the size of the shorts body, and is 10 to 35 cm, preferably 25 to 30 cm. The width of the rear end part 22 is 7 to 20 cm, preferably 10 to 15 cm.

Then, as shown in FIG. 3B, the predetermined length part (trapezoidal part) 28 from the front end part 21 to the center corresponding to the crotch part can be composed of a cloth material different from the other parts. Preferably, the cloth of the trapezoidal part 28 can be composed of a cloth material having excellent heat-retaining property and elasticity. Specifically, it may be a knit made of cotton, animal hair, or the like, and a material having a heat retaining effect such as terahertz ore may be mixed. The length of the trapezoidal portion 28 in the front-rear direction is 5 to 20 cm, preferably 10 to 16 cm. The trapezoidal part 28 is a part that makes contact with the lower abdomen when worn, and by using a material with a wide width and excellent heat-retaining property, the abdomen can be warmed, which is particularly comfortable for women during menstruating. The trapezoidal part 28 can be substantially made of a heat-retaining material, and it is not necessary to use a material having high water absorption and water retention.

Next, the absorption member 2 preferably has a structure in which the first cloth member 25 and the second cloth member 26 are laminated on the rear side from the trapezoidal part 28. The first cloth member 25 and the second cloth member 26 can have substantially the same dimensions. FIG. 3C shows the shape of the first cloth member 25. The first cloth member 25 comes into direct contact with the skin when worn. The first cloth member 25 is sewn and joined to the rear end part of the trapezoidal part 28 at the front end part. If the trapezoidal part 28 is not made of a different material, the first cloth member 25 may be arranged up to the front end part 21. Further, in the portion corresponding to the crotch part, it has a portion making width narrower (narrow width part 251). The first cloth member 25 may be formed by laminating several pieces of cloth as needed.

FIG. 3D shows the shape of the second cloth member 26. The second cloth member 26 is laminated below the first cloth member 25 and arranged between the first cloth member 25 and the shorts body 3. The front end part of the second cloth member 26 is sewn and joined to the rear end part of the trapezoidal part 28. If the trapezoidal part 28 is not made of a different material, the second cloth member 26 may be arranged up to the front end part 21. It has wing parts 23 for side to side in the portion corresponding to the crotch part. The wing part 23 has a width W1 of 2 to 15 cm, preferably 5 to 10 cm, in the front-rear direction. The width W1 refers to the width at the end part 231 in the left and right direction of the wing part 23 as shown in FIG. 3D. The length W2 of the wing part 23 in the left and right direction is 2 to 5 cm, preferably 3 to 4 cm.

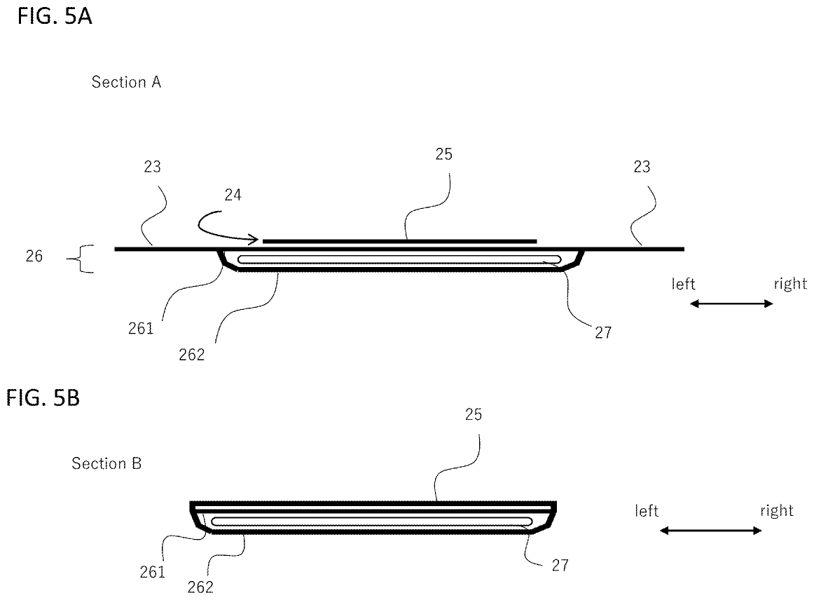

The second cloth member 26 has a water-absorbing material at least in the central part in the left and right direction. As an example, the water-absorbing material 27 can be arranged in the portion shown by the dotted line in FIG. 3D. For example, as shown in FIG. 5, the second cloth member 26 may be composed of a combined body in which the first cloth 261 on the skin side, the water-absorbing material 27, and the second cloth 262 on the shorts body 3 side are laminated, and the periphery thereof is sewn together. The water-absorbing material 27 is provided at least in a portion of the second cloth member 26 that corresponds to the crotch part. Preferably, as shown in FIG. 3D, the second cloth member 26 is present over almost the entire length in the front and rear direction. That is, since the rear end part 22 of the second cloth member 26 is connected to the upper end part (waist rubber) 36 of the back body part of the shorts body 3, the water absorbing material is arranged over almost the entire length of the second cloth member 26. Thereby, even if the liquid flows to the rear when lying down, it can be absorbed.

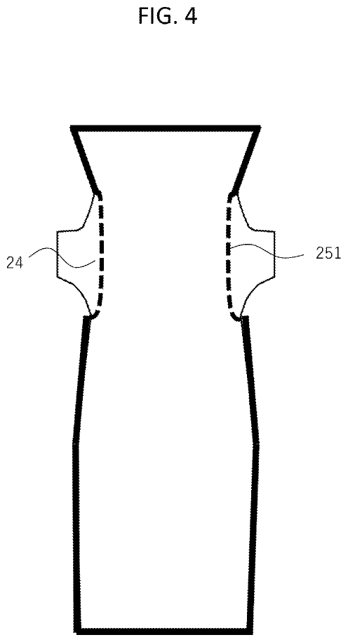

The first cloth member 25 and the second cloth member 26 are laminated so that the first cloth member 25 is on the skin side, and the periphery thereof is sewn and joined to each other. At this time, at least a predetermined portion of the first cloth member 25 in the portion corresponding to the crotch part is in a separable state without being sewn with the second cloth member 26. Preferably, the narrow width part 251 of the first cloth member 25 is not sewn with the second cloth member 26. FIG. 4 is a view showing a stitching portion between the first cloth member 25 and the second cloth member 26. The first cloth member 25 and the second cloth member 26 are sewn together at their ends at the thick solid line portion in the figure when they are overlapped. On the other hand, they are no sewn together at the dotted line. Therefore, in the dotted line portion that is not sewn, a pocket 24 is formed between the first cloth member 25 and the second cloth member 26. By forming the pocket 24, when a commercially available sanitary napkin is attached onto the first cloth member 25, the so-called wing part of the sanitary napkin can be folded into the pocket 24 to prevent the sanitary napkin from deviating. A commercially available sanitary napkin can also be installed in the space between the first cloth member 25 and the second cloth member 26. By arranging a commercially available sanitary napkin between the first cloth member 25 and the second cloth member 26, the sanitary napkin does not come into direct contact with the skin. Therefore, even people who are prone to skin irritation from sanitary napkins can use it with confidence.

FIGS. 5A and 5B is a cross-sectional view of the absorbing member 2. FIG. 5A is a cross-sectional view taken along the line A in FIG. 3A. FIG. 5B is a cross-sectional view taken along the line B in FIG. 3A. In FIG. 5A, first, the second cloth member 26 has at least a three-layer structure of the first cloth 261 arranged on the skin side, the absorbing material 27, and the second cloth 262 arranged on the shorts body 3 side. The absorbing material 27 has a width slightly shorter than the width of the second cloth member 25 in the left-right direction. The second fabric 262 and the first fabric 261 are sewn around the absorbing material 27 to be held between the first fabric 261 and the second fabric 262. However, the present disclosure is not limited thereto, and the end part of the absorbing material 27 may be sewn with the first cloth 261 or the second cloth 262. For example, if the peripheral edge part of the absorbing material 27 is sewn to the end part of the first fabric 261, the position of the absorbent material 27 is fixed, which is preferable. In the cross-sectional view taken along line A, the first cloth member 25 is not sewn to the second cloth member 26 and forms a pocket 24 in the end part.

In FIG. 5B, the first cloth member 25 and the second cloth member 26 are sewn together at the end parts in the left and right direction. Specifically, the first cloth member 25, and the first cloth 261 and the second cloth 262 of the second cloth member may be sewn together at the end parts of the left and right direction. Since the first cloth member 25 and the second cloth member 26 are not sewn onto the surface part other than the end part, there is a space between the two, and a sanitary napkin can be inserted from the pocket 24 as described above. Further, the end part may be provided with a folded portion in order to prevent the liquid from causing side leakage.

When the second cloth member 26 has a three-layer structure of the first cloth 261, the absorbing material 27 and the second cloth 262, each material can be as follows. First, the first fabric 261 is preferably a material having high elasticity without rewetting and having high liquid permeability in order to allow the liquid to pass through the absorbing material 27 in the lower layer, and a polyester mesh or the like can be used. Further, the absorbing material 27 is a material 1s having liquid absorbency, liquid diffusivity, and liquid maintainability, and examples thereof include cotton, cotton pile, antibacterial and deodorized acrylic, water-absorbing acrylic, water-absorbing polyester, cupra, rayon and other knits and non-woven fabrics, particularly, a mixed material consisting of rayon and acrylic. The water absorption capacity of the absorbing material 27 can be appropriately adjusted according to the degree of menstrual blood and the amount of urine. For example, it is preferable to be able to absorb 10 ml to 300 ml of liquid. The second fabric 262 is preferably made of a waterproof and moisture permeable material, for example, polyester, and those which is further laminated with polyurethane film, or alternatively, a film such as polyurethane, polyethylene or polyvinyl chloride can be used. By providing the second fabric 262 having waterproof property, it is possible to prevent the liquid from seeping into the shorts body 3.

Further, since the first cloth member 25 comes into direct contact with the skin when worn, a material that is elastic without rewetting and has high liquid permeability to allow liquid to pass through the lower layer is preferable, and polyester mesh or the like can be used. The fabrics comprising the first cloth member 25 and the second cloth member 26 may be subjected to antibacterial and deodorization processing, if necessary.

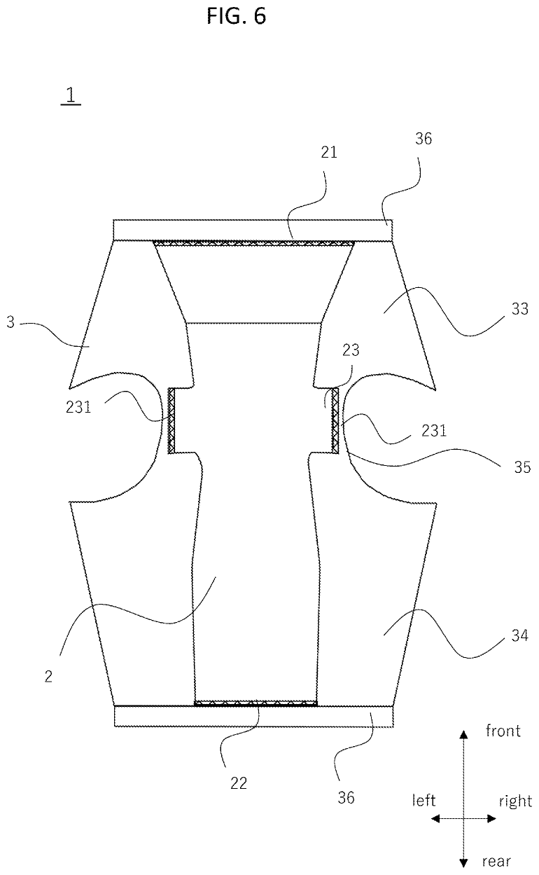

FIG. 6 is a view showing a modified example of the absorption shorts of the present disclosure. This modified example differs from the above example in that it does not have the first cloth member 25. Therefore, the absorbing member 2 is substantially comprising a laminated structure of the first cloth 261, the absorbing material 27 and the second cloth 262. In this case, a pocket 24 is not formed. Further, in the same manner as in the first embodiment, the predetermined to length part (trapezoidal part) 28 may be made of a different material having a heat retaining effect from the front end part 21 to the central portion corresponding to the crotch part. According to the modified example, the cost can be suppressed as the number of parts is reduced. Moreover, since the absorption shorts of the present disclosure can guarantee a sufficient amount of water supply, the variant can be used for those who do not need to further use a commercially available sanitary napkin.

According to the absorption shorts of the present disclosure, since the absorption member 2 having high water absorption is attached, menstrual blood and urine can be absorbed without attaching a sanitary napkin or an incontinence pad. In the absorption shorts of the present disclosure, the absorption member 2 is sewn onto the shorts body 3 at the front end part 21, the rear end part 22, and the wing part 23, and is separable at the other parts, so that the absorption member 2 can be easily pulled out when washing by rubbing.

Also, if a wearer wants to use a sanitary napkin or incontinence pad for more peace of mind, since the pocket 24 is formed between the first cloth member 25 and the second cloth member 26, a sanitary napkin or the like can be put in the pocket 24. When mounted on the first cloth member 25, it can be easily fixed by inserting the wings of the sanitary napkin into the pocket 24.

The above-described embodiments are merely examples for facilitating the understanding of the present disclosure, and should not be construed as limiting the present disclosure. It goes without saying that modification and improvement can be made to the present disclosure without departing from the spirit thereof, and that an equivalent thereof is included in the present disclosure.

### DESCRIPTION OF REFERENCE NUMERALS

1: absorption shorts
2: water absorbing part
3: shorts body
21: front end part
22: rear end part
23: wing part
24: pocket
25: first cloth member
26: second cloth member
27: absorption material
28: the trapezoidal part
31: waist opening
32: leg opening
33: front body part
34: back body part
35: crotch part
36: waist rubber

1. An absorption shorts comprising:
a shorts body with a waist opening and a pair of leg openings; and
an absorption member,
wherein the absorption member is sewn onto the shorts body only at a front end part, a rear end part, and a pair of wing parts on the left and right sides in the portion corresponding to the crotch part,
wherein the absorption member has a fabric member comprising at least three or more layers: a first fabric on the skin side; absorbing material; and a second fabric on the shorts body side, and the first fabric and the second fabric are sewn together at the edges.

2. The absorption shorts according to claim 1,
wherein the length of the wings in the left-right direction is 2 to 5 cm.

3.-9. (canceled)

10. The absorption shorts according to claim 1,
wherein the absorbing material is capable of absorbing equal to or more than 10 ml of liquid.

11. The absorption shorts according to claim 2,
wherein the absorbing material is capable of absorbing equal to or more than 10 ml of liquid.

12. The absorption shorts as in any one of claim 1,
wherein the first cloth is a mesh material, and the second cloth is a waterproof material.

13. The absorption shorts as in any one of claim 2,
wherein the first cloth is a mesh material, and the second cloth is a waterproof material.

14. The absorption shorts as in any one of claim 10,
wherein the first cloth is a mesh material, and the second cloth is a waterproof material.

15. The absorption shorts as in any one of claim 11,
wherein the first cloth is a mesh material, and the second cloth is a waterproof material.

16. The absorption shorts as in one of claim 1,
wherein at least the front end part and the rear end part of the absorption member is sewn to the shorts body along a waist rubber of the shorts body.

17. The absorption shorts as in one of claim 2,
wherein at least the front end part and the rear end part of the absorption member is sewn to the shorts body along a waist rubber of the shorts body.

18. The absorption shorts as in one of claim 10,
wherein at least the front end part and the rear end part of the absorption member is sewn to the shorts body along a waist rubber of the shorts body.

19. The absorption shorts as in one of claim 11,
wherein at least the front end part and the rear end part of the absorption member is sewn to the shorts body along a waist rubber of the shorts body.

20. The absorption shorts as in one of claim 12,
wherein at least the front end part and the rear end part of the absorption member is sewn to the shorts body along a waist rubber of the shorts body.

21. The absorption shorts as in one of claim 13,
wherein at least the front end part and the rear end part of the absorption member is sewn to the shorts body along a waist rubber of the shorts body.

22. The absorption shorts as in one of claim 14,
wherein at least the front end part and the rear end part of the absorption member is sewn to the shorts body along a waist rubber of the shorts body.

23. The absorption shorts according to claim 1,
wherein the absorption member has a heat retaining part from the front end part to a lower abdomen.

24.-27. (canceled)
